(19) United States
(12) Patent Application Publication
(10) Pub. No.: US 2013/0325068 A1
(43) Pub. Date: Dec. 5, 2013

(54) FLEXION LIMITING DEVICE WITH SPINOUS PROCESS ATTACHMENT

(71) Applicant: Simpirica Spine, Inc., San Carlos, CA (US)

(72) Inventors: Louis Fielding, San Carlos, CA (US); Hugues Malandain, Mountain View, CA (US)

(73) Assignee: Simpirica Spine, Inc., San Carlos, CA (US)

(21) Appl. No.: 13/963,797

(22) Filed: Aug. 9, 2013

Related U.S. Application Data
(63) Continuation of application No. PCT/US2012/026254, filed on Feb. 23, 2012.
(60) Provisional application No. 61/445,930, filed on Feb. 23, 2011.

(51) Int. Cl. A61B 17/70; A61B 17/88; A61B 17/84

(52) U.S. Cl. CPC A61B 17/7062; A61B 17/842; A61B 17/8861
USPC 606/263; 606/279; 606/86 A

(57) ABSTRACT

A device for restricting flexion of a spine comprises a pair of compliance members that are disposed around a spinal midline, an upper fastener, and a lower fastener. The upper fastener is configured to be coupled to a superior spinous process and the lower fastener is configured to be coupled to an inferior spinous process. The pair of compliance members are coupled to the upper and the lower fasteners such that the compliance members provide a force resistant to flexion between the upper and the lower spinous processes.

### CROSS-REFERENCE

The present application is a continuation of International PCT Patent Application No. PCT/US2012/026254 (Attorney Docket No. 41564-720.601) filed Feb. 23, 2012 which claims the benefit of U.S. Provisional Application No. 61/445,930 (Attorney Docket No. 41564-720.101), filed Feb. 23, 2011; the entire contents of which is incorporated herein by reference.

This application is also related to the following U.S. patent application Ser. Nos. 12/106,049, 12/106,103, 12/479,016, and 13/037,039, and 13/267,394; U.S. Provisional Patent Application No. 60/936,897; and the following U.S. Patent Publication Nos. 2008/0009866 and 2008/0108993; the entire contents of which are incorporated herein by reference.

### BACKGROUND OF THE INVENTION

1. Field of the Invention

The present invention generally relates to medical methods and apparatus. More particularly, the present invention relates to orthopedic internal fixation such as methods, devices, and accessories for restricting spinal flexion in patients having back pain or instability, or other orthopedic applications where internal fixation may be employed and other uses that the fixation structure may advantageously provide.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine. Pain tends to be exacerbated when patients put their lumbar spines into flexion (i.e. by sitting or bending forward) and relieved when they put their lumbar spines into extension (i.e. by standing or arching backwards). Flexion and extension are known to change the mechanical loading pattern of a lumbar segment. When the segment is in extension, the axial loads borne by the segment are shared by the disc and facet joints (approximately 30% of the load is borne by the facet joints). In flexion, the segmental load is borne almost entirely by the disc. Furthermore, the nucleus shifts posteriorly, changing the loads on the posterior portion of the annulus (which is innervated), likely causing its fibers to be subject to tension and shear forces. Segmental flexion, then, increases both the loads borne by the disc and causes them to be borne in a more painful way. Discogenic pain can be quite disabling, and for some patients, can dramatically affect their ability to work and otherwise enjoy their lives.

Pain experienced by patients with discogenic low back pain can be thought of as flexion instability, and is related to flexion instability manifested in other conditions. The most prevalent of these is spondylolisthesis, a spinal condition in which abnormal segmental translation is exacerbated by segmental flexion. Flexion instability may be surgically-induced during common procedures such as neural decompression for spinal stenosis. This iatrogenic flexion instability may lead to back pain or recurrence of neurological symptoms. The methods and devices described should as such also be useful for these other spinal disorders or treatments associated with segmental flexion, for which the prevention or control of spinal segmental flexion is desired. Another application for which the methods and devices described herein may be used is in conjunction with a spinal fusion, in order to restrict motion, promote graft fusion and healing, and relieve pain post-operatively. Alternatively, the methods and devices described should also be useful in conjunction with other treatments of the anterior column of the spine, including kyphoplasty, total disc replacement, nucleus augmentation and annular repair. General orthopedic or surgical applications are envisioned where screw, rod, or plate fixation, or a tether, cable or tape may be employed.

Patients with discogenic pain accommodate their syndrome by avoiding positions such as sitting, which cause their painful segment to go into flexion, preferring positions such as standing, which maintain their painful segment in extension. One approach to reducing discogenic pain involves the use of a lumbar support pillow often seen in office chairs. Biomechanically, the attempted effect of the ubiquitous lumbar support pillow is also to maintain the painful lumbar segment in the less painful extension position. Postural and muscular compensation for spinal instability involves significant recruitment of the paraspinal musculature, and may exacerbate back pain.

Current treatment alternatives for patients diagnosed with chronic discogenic pain or flexion instability are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion may or may not also include instrumentation of the affected spinal segment including, for example, pedicle screws and stabilization rods. Fusion is not usually recommended for discogenic pain because it is irreversible, costly, associated with high morbidity, and has questionable effectiveness. Despite its drawbacks, however, spinal fusion for discogenic pain remains common due to the lack of viable alternatives.

An alternative method, that is not commonly used in practice, but has been approved for use by the United States Food and Drug Administration (FDA), is the application of bone cerclage devices which can encircle the spinous processes or other vertebral elements and thereby create a restraint to motion. Physicians typically apply a tension or elongation to the devices that applies a constant and high force on the anatomy, thereby fixing the segment in one position and allowing effectively no motion. The lack of motion allowed after the application of such devices is thought useful to improve the likelihood of fusion performed concomitantly; if the fusion does not take, these devices will fail through breakage of the device or of the spinous process to which the device is attached. These devices are designed for static applications and are not designed to allow for dynamic elastic resistance to flexion across a range of motion. The purpose of bone cerclage devices and other techniques described above is to almost completely restrict measurable motion of the vertebral segment of interest. This loss of motion at a given segment gives rise to abnormal loading and motion at adjacent segments, which can lead eventually to adjacent segment morbidity.

Another solution involves the use of an elastic structure coupled to the spinal segment. The elastic structures are typically secured to the spinal segment with pedicle screws, or sometimes tethers. The elastic structures can relieve pain by increasing passive resistance to flexion while often allowing substantially unrestricted spinal extension. This mimics the mechanical effect of postural accommodations that patients already use to provide relief.

Spinal implants using elastic structures are currently commercially available. One such implant couples adjacent vertebrae via their pedicles. This implant includes flexible couplers and pedicle screws. To install the implant, selected portions of the disc and vertebrae bone are removed. Implants are then placed to couple two adjacent pedicles on each side of the spine. The pedicle screws secure the implants in place. The elastic coupler is secured to the pedicle screws, and limits the extension/flexion movements of the vertebrae of interest. Because significant tissue is removed and because of screw placement into the pedicles, the implant and accompanying surgical methods are highly invasive and the implant is often irreversibly implanted. There is also an accompanying high chance of nerve root damage.

Other elastic implants employ tether structures to couple adjacent vertebrae via their processes instead. These implants include a tether and a spacer. To install the implant, the supraspinous ligament is temporarily lifted and displaced. The interspinous ligament between the two adjacent vertebrae of interest is then permanently removed and the spacer is inserted in the interspinous interspace. The tether is then wrapped around the processes of the two adjacent vertebrae, through adjacent interspinous ligaments, and then mechanically secured in place by the spacer or also by a separate component fastened to the spacer. The supraspinous ligament may then be restored back to its original position. Such implants and accompanying surgical methods are not without disadvantages. These implants may subject the spinous processes to frequent, high loads during everyday activities, sometimes causing the spinous processes to break or erode. Furthermore, the spacer may put a patient into segmental kyphosis, potentially leading to long-term clinical problems associated with lack of sagittal balance. The process of securing the tethers is often a very complicated maneuver for a surgeon to perform, making the surgery much more invasive. And, as previously mentioned, the removal of the interspinous ligament is permanent. As such, the application of the device is not reversible.

More recently, less invasive spinal implants have been introduced. Like the aforementioned implant, these spinal implants are placed over one or more pairs of spinous processes and provide an elastic restraint to the spreading apart of the spinous processes during flexion. However, spacers are not used and interspinous ligaments are not permanently removed. As such, these implants are less invasive and may be reversibly implanted. The implants typically include a tether and a securing mechanism for the tether. The tether may be made from a flexible polymeric textile such as woven polyester (PET) or polyethylene; multi-strand cable, or other flexible structure. The tether is wrapped around the processes of adjacent vertebrae and then secured by the securing mechanism. The securing mechanism may involve the indexing of the tether and the strap, e.g., the tether and the securing mechanism include discrete interfaces such as teeth, hooks, loops, etc. which interlock the two. Highly forceful clamping may also be used to press and interlock the tether with the securing mechanism. Many known implementations can clamp a tether with the tip of a set-screw, or the threaded portion of a fastener. However, the mechanical forces placed on the spinal implant are unevenly distributed towards the specific portions of the tether and the securing mechanism which interface with each other. These portions are therefore typically more susceptible to abrasion, wear, or other damage, thus reducing the reliability of these spinal implants as a whole. Other known methods use a screw or bolt to draw other components together to generate a clamping force. While these methods may avoid the potentially damaging loads, the mechanical complexity of the assembly is increased by introducing more subcomponents. Other methods use a buckle through which the tether is threaded in a tortuous path, creating sufficient friction to retain the tether. These buckles generally distribute the load over a length of the tether; although they may be cumbersome to use and adjust as the tether is required to be threaded around multiple surfaces and through multiple apertures. Many of the aforementioned methods involve the use of several components, which must often be assembled during the surgical procedure, often within the wound. This adds time, complexity and risk to the surgical procedure.

The less invasive tether devices described above are promising for treating pain or instability related to flexion of the lumbar spine. However, in some cases, the tether may slip off the processes, the tether may not be securely fastened thereto, or the procedure of placing and securing the tether may be cumbersome. Therefore it would be desirable to provide a device for restricting flexion that has improved attachment to a spinous process or other portion of the vertebra. Such a device should be easy to implant, have components that do not interfere with one another, and preferably is minimally invasive. Also, such methods and apparatus should preferably enable the implant to be more easily, reversibly, repeatably, safely and reliably be implanted and adjusted by a surgeon, in a surgery setting. At least some of these objectives will be met by the exemplary embodiments disclosed herein.

2. Description of the Background Art

Patents and published applications of interest include: U.S. Pat. Nos. 3,648,691; 4,643,178; 4,743,260; 4,966,600; 5,011,494; 5,092,866; 5,116,340; 5,180,393; 5,282,863; 5,395,374; 5,415,658; 5,415,661; 5,449,361; 5,456,722; 5,462,542; 5,496,318; 5,540,698; 5,562,737; 5,609,634; 5,628,756; 5,645,599; 5,725,582; 5,902,305; Re. 36,221; 5,928,232; 5,935,133; 5,964,769; 5,989,256; 6,053,921; 6,248,106; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; 7,029,475; 7,163,558; Published U.S. Patent Application Nos. US 2002/0151978; US 2004/0024458; US 2004/0106995; US 2004/0116927; US 2004/0117017; US 2004/0127989; US 2004/0172132; US 2004/0243239; US 2005/0033435; US 2005/0049708; 2005/0192581; 2005/0216017; US 2006/0069447; US 2006/0136060; US 2006/0240533; US 2007/0213829; US 2007/0233096; Published PCT Application Nos. WO 01/28442 A1; WO 02/03882 A2; WO 02/051326 A1; WO 02/071960 A1; WO 03/045262 A1; WO2004/052246 A1; WO 2004/073532 A1; and Published Foreign Application Nos. EP0322334 A1; and FR 2 681 525 A1. The mechanical properties of flexible constraints applied to spinal segments are described in Papp et al. (1997) Spine 22:151-155; Dickman et al. (1997) Spine 22:596-604; and Garner et al. (2002) Eur. Spine J. S186-S191; Al Baz et al. (1995) Spine 20, No. 11, 1241-1244; Heller, (1997) Arch. Orthopedic and Trauma Surgery, 117, No. 1-2:96-99; Leahy et al. (2000) Proc. Inst. Mech. Eng. Part H: J. Eng. Med. 214, No. 5: 489-495; Minns et al., (1997) Spine 22 No. 16:1819-1825; Miyasaka et al. (2000) Spine 25, No. 6: 732-737; Shepherd et al. (2000) Spine 25, No. 3: 319-323; Shepherd (2001) Medical Eng. Phys. 23, No. 2: 135-141; and Voydeville et al (1992) Orthop Traumatol 2:259-264.

### BRIEF SUMMARY OF THE INVENTION

The present invention provides methods and apparatus that may be used to restrict flexion of the spine. Such devices are preferably coupled to the spinous processes in a secure but minimally invasive manner. These devices may provide a promising treatment for flexion-related pain, such as discogenic pain, as well as other conditions involving flexion-related pain or instability, such as degenerative spondylolisthesis.

In a first aspect of the present invention, a device for restricting flexion of a spine is provided. This device comprises at least one compliance member, an upper fastener, and a lower fastener. The at least one compliance member main comprise a pair of compliance members configured to be disposed across a spinal midline. The upper fastener is configured to be fixedly coupled to a superior spinous process, and the lower fastener is configured to be fixedly coupled to an inferior spinous process. The at least one compliance member is configured to be coupled to the upper and lower fasteners such that the compliance members provide a force resistant to flexion between the upper and the lower spinous processes.

The compliance members may be configured in various ways. One or more of the compliance members may comprise an elastic member configured to provide an elastic tension between the superior spinous process and inferior spinous process to resist flexion therebetween. The elastic member may, for example, be a spring. A compliance member may comprise a hollow, cylindrical main body and the elastic member may comprise a spring cut into the cylindrical main body.

One or more of the compliance members may be configured to be laterally flexible so that axial rotation and lateral bending of the spine are generally unrestricted when the device is attached to the spine, i.e., when the upper fastener is coupled to the superior spinous process, the lower fastener is coupled to the inferior spinous process, and the pair of compliance members are coupled to the lower and upper fasteners.

The axial length of the device can be adjusted, e.g., to accommodate the variety of distances between spinous processes. To adjust the axial length of the device, the length of one or more of the compliance members may be adjustable, e.g., to provide a selectable amount of tension.

The upper and lower fasteners may be configured in various ways. In many embodiments, the upper and/or lower fasteners are monolithic and resilient such that the fastener can be clipped onto the superior and/or inferior spinous process, respectively. Additionally or in the alternative, one or more protrusions can be provided on the interior surfaces of the fasteners to facilitate fixed attachment of the fastener to a spinous process. The interior surfaces of the fasteners may also in many embodiments be treated to facilitate osseointegration with the spinous process, e.g., by treatment with a plasma bead spray, hydroxyapatite, etc. In many embodiments, the fastener may comprise a screw, bolt, pin, or the like that is passed through the spinous process.

The device may be configured in various ways so that tensile load can be transmitted to the compliance members while preventing compressive and/or other loads (e.g., bending, torsional, sheer loads, etc.) from being transmitted thereto. For example, the compliance members may comprise a telescoping column, flexible tether structures may connect the compliance members with the fasteners, or non-compliant connectors may connect the compliance members with the fasteners through universal joints such as ball joints, Cardan joints, or flexible elastomeric couplings (e.g., Boge joints).

The at least one compliance member may provide an elastic resistance to flexion in a range from 7.5 N/mm to 20 N/mm, preferably 10 N/mm to 15 N/mm, so that effective pain relief can be achieved with minimum risk of damage to the spinous processes and other vertebral and spinal structures which could result from restriction with relatively rigid structures and even elastic structures with higher elastic resistances.

Another aspect of the present invention provides a method for restricting flexion of a spine. An upper fastener is attached to an upper spinous process. A lower fastener is attached to a lower spinous process. At least one compliance member is coupled to both the upper and lower fasteners. Flexion is resisted between the upper and lower spinous processes. The at least one compliance members provides a force resistant to the flexion. Between the upper and lower spinous processes, there may be no other spinous processes or one or more spinous process. In many embodiments, bending, torsional, or shear loads are prevented from being transferred to the compliance members while tensile loads are allowed to be transferred to the compliance members. The at least one compliance member may be assembled with the upper and lower fasteners during surgery. Or, the at least one compliance member may come pre-assembled with the upper and/or lower fastener. Typically, the at least one compliance member will comprise a first compliance member and a second compliance member, the first and second compliance members being disposed on opposite sides of a spinal midline.

A fastener, upper or lower, may be attached to a spinous process, upper or lower, in various ways. A fastener may be crimped over a spinous process such that the fastener is fixedly secure thereto. A fastener may comprise an adjustable clamping mechanism which may be tightened as the fastener is placed over a spinous process to fixedly secure the fastener thereto. A fastener may be resilient and biased such that it need be expanded, placed in its expanded form over a spinous process, and released from expansion so that a spring force in the fastener causes it to clip over the spinous process.

In many embodiments, tools and methods are further provided to facilitate the attachment of the fasteners to the spinal processes. A thickness measurement and crimping tool may be provided. The tool comprises a pair of handles, a pair of jaws, a ruler disposed between proximal ends of the pair of handles, and a moveable stop disposed on the ruler. Before any attachment of a fastener to a spinous process, the thickness of a spinous process of interest is measured by clenching the pair of jaws of tools over the spinous process. A maximum closure distance of the jaws can be set by adjusting the location of the moveable stop on the ruler. The maximum closure distance corresponds to the measured thickness of the upper spinous process. The fastener is then positioned over the spinous process and crimped with the pair of jaws such that the fastener is fixedly secure to the spinous process without substantial damage to the bone of the spinous process, e.g., undesired bone crushing, bone fracture, bone contusions, etc.

In many embodiments, an elastic resistance to flexion in a range from 7.5 N/mm to 20 N/mm, preferably 10 N/mm to 15 N/mm, is provided to resist flexion so that effective pain relief can be achieved with minimum risk of damage to the spinous processes and other vertebral and spinal structures which could result from restriction with relatively rigid structures and even elastic structures with higher elastic resistances.

Yet another aspect of the present invention provides a system for restricting flexion of a spine. The system includes the spinal flexion limiting device described above and a thickness measurement and crimping tool. Again, the spinal flexion limiting device comprises a pair of compliance members, an upper fastener, and a lower fastener. The pair of compliance members is configured to be disposed across a spinal midline. The upper fastener is configured to be fixedly coupled to a superior spinous process, and the lower fastener is configured to be fixedly coupled to an inferior spinous process. The pair of compliance members are configured to be coupled to the upper and lower fasteners such that the compliance members provide a force resistant to flexion between the upper and the lower spinous processes. The thickness measurement and crimping tool comprises a pair of handles, a pair of jaws, a ruler disposed between the proximal ends of the pair of handles, and a moveable stop disposed on the ruler. The pair of jaws is configured for measuring the thickness of a spinous process. The moveable stop is configured to limit the closure distance of the pair of jaws such that the lower or upper fastener of the device can be crimped over the inferior or superior spinous process by the pair of jaws without substantial damage to the bone of said spinous process, e.g., undesired bone crushing, bone fracture, bone contusions, etc.

These and other embodiments are described in further detail in the following description related to the appended drawing figures.

### BRIEF DESCRIPTION OF THE DRAWINGS

FIG. 1 is a schematic diagram illustrating the lumbar region of the spine.

FIG. 1A a schematic illustration showing a portion of the lumbar region of the spine taken along a sagittal plane.

FIG. 2 illustrates a spinal implant of the type described in US 2005/0216017A1.

FIGS. 3A-3B illustrate additional tissue surrounding the spinous processes.

FIGS. 4A-4M show an exemplary method of surgically implanting a spinal device.

FIG. 5 illustrates an exemplary compliance element.

FIGS. 6A-6C illustrate the use of an exemplary fastening mechanism incorporated in the compliance element for removably locking a tether.

FIG. 7 is an exploded view of an exemplary fastening mechanism.

FIGS. 8A and 8A1 are side views of exemplary spinal implants attached to a spine.

FIGS. 8B and 8B1 are posterior views of the spinal implant of FIGS. 8A and 8A1 attached to a spine.

FIGS. 8C-8E show partial cross-sectional views of various embodiments of the attachment member of the spinal implant of FIGS. 8A-8B.

FIGS. 9A-9C illustrate exemplary spinal implants according to various embodiments of the invention.

FIGS. 9D-9E show cross-sectional views of the telescoping mechanism of the spinal implant of FIG. 9C.

FIGS. 10A-10B illustrate an exemplary spinal process thickness measurement and spinal implant attachment member crimping tool.

### DETAILED DESCRIPTION OF THE INVENTION

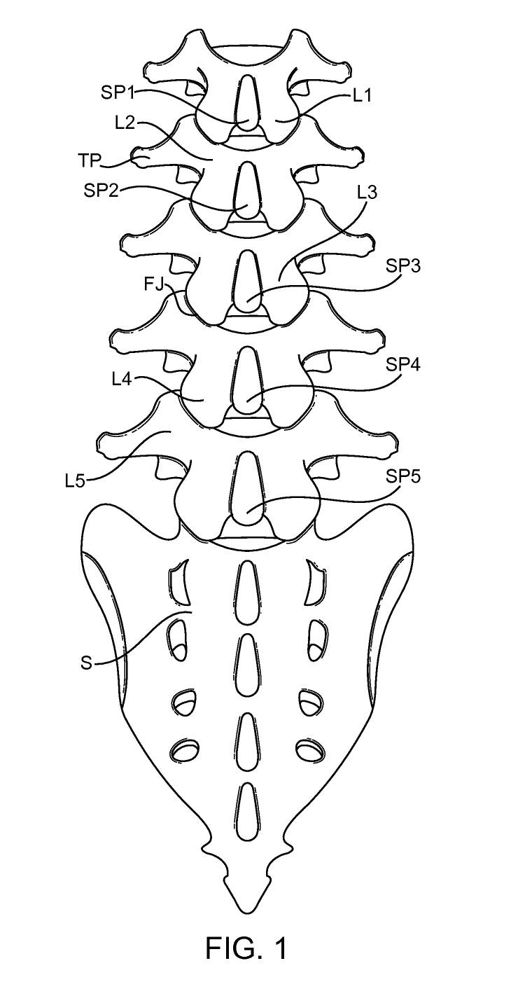

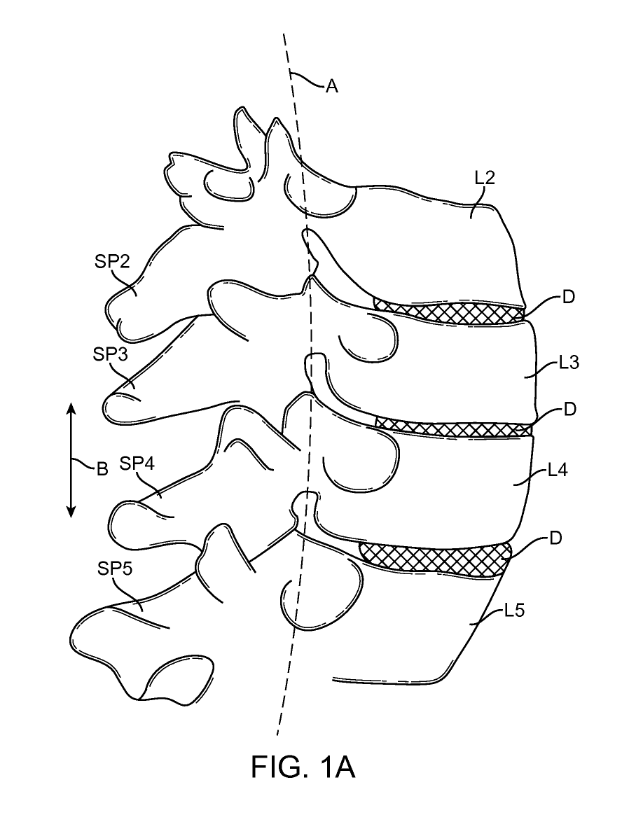

FIG. 1 is a schematic diagram illustrating the lumbar region of the spine including the spinous processes (SP), facet joints (FJ), lamina (L), transverse processes (TP), and sacrum (S). FIG. 1A is a schematic illustration showing a portion of the lumbar region of the spine taken along a sagittal plane and is useful for defining the terms “neutral position,” “flexion,” and “extension” that are often used in this disclosure.

As used herein, “neutral position” refers to the position in which the patient's spine rests in a relaxed standing position. The “neutral position” will vary from patient to patient. Usually, such a neutral position will be characterized by a slight curvature or lordosis of the lumbar spine where the spine has a slight anterior convexity and slight posterior concavity. In some cases, the presence of the constraint of the present invention may modify the neutral position, e.g. the device may apply an initial force which defines a “new” neutral position having some extension of the untreated spine. As such, the use of the term “neutral position” is to be taken in context of the presence or absence of the device. As used herein, “neutral position of the spinal segment” refers to the position of a spinal segment when the spine is in the neutral position.

Furthermore, as used herein, “flexion” refers to the motion between adjacent vertebrae in a spinal segment as the patient bends forward. Referring to FIG. 1A, as a patient bends forward from the neutral position of the spine, i.e. to the right relative to a curved axis A, the distance between individual vertebrae L on the anterior side decreases so that the anterior portion of the intervertebral disks D are compressed. In contrast, the individual spinous processes SP on the posterior side move apart in the direction indicated by arrow B. Flexion thus refers to the relative movement between adjacent vertebrae as the patient bends forward from the neutral position illustrated in FIG. 1A.

Additionally, as used herein, “extension” refers to the motion of the individual vertebrae L as the patient bends backward and the spine extends from the neutral position illustrated in FIG. 1A. As the patient bends backward, the anterior ends of the individual vertebrae will move apart. The individual spinous processes SP on adjacent vertebrae will move closer together in a direction opposite to that indicated by arrow B.

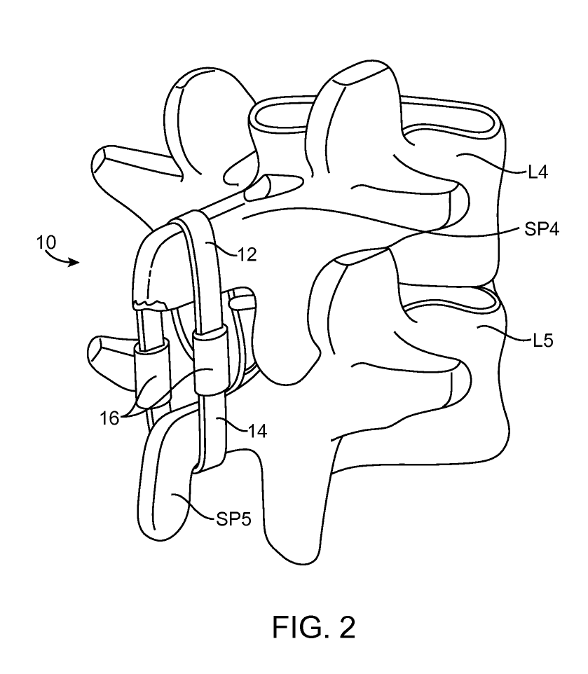

FIG. 2 shows a spinal implant of the type described in related U.S. Patent Publication No. 2005/0216017 A1 (now U.S. Pat. No. 7,458,981), the contents of which are herein incorporated by reference. As illustrated in FIG. 2, an implant 10 typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring or rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinous processes which provides a force that resists flexion. The force increases as the processes move further apart. Usually, the straps themselves will be essentially non-compliant so that the degree of elasticity or compliance may be controlled and provided solely by the compliance members 16.

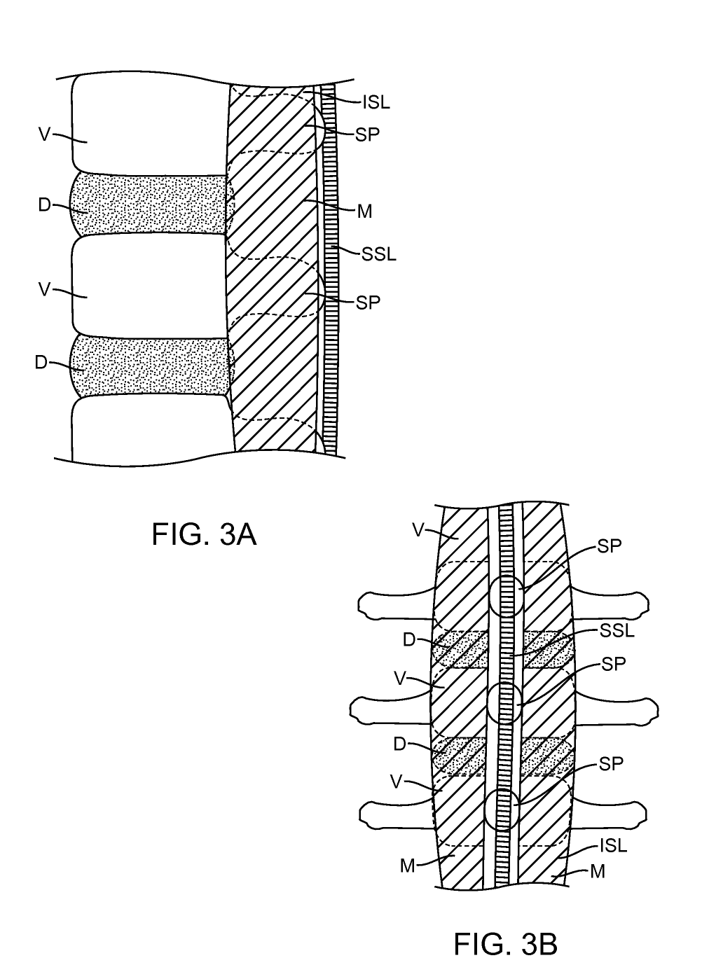

FIG. 3A is a side view of the lumbar region of the spine having discs D separating the vertebral bodies V. The supraspinous ligament SSL runs along the posterior portion of the spinous processes SP and the interspinous ligament ISL and multifidus tendon and muscle M run alongside of and attach to the spinous processes SP. FIG. 3B is a posterior view of FIG. 3A.

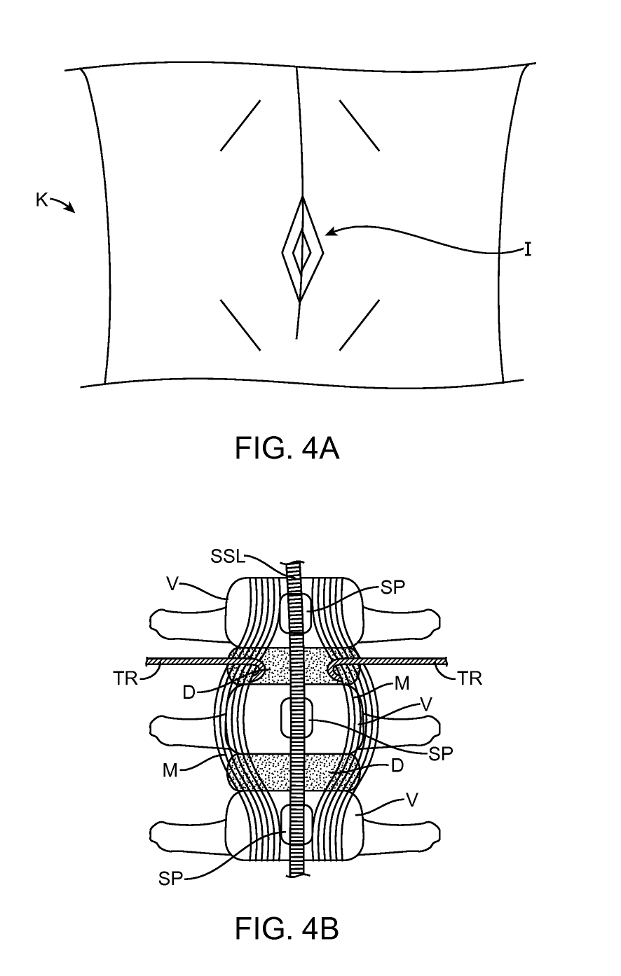

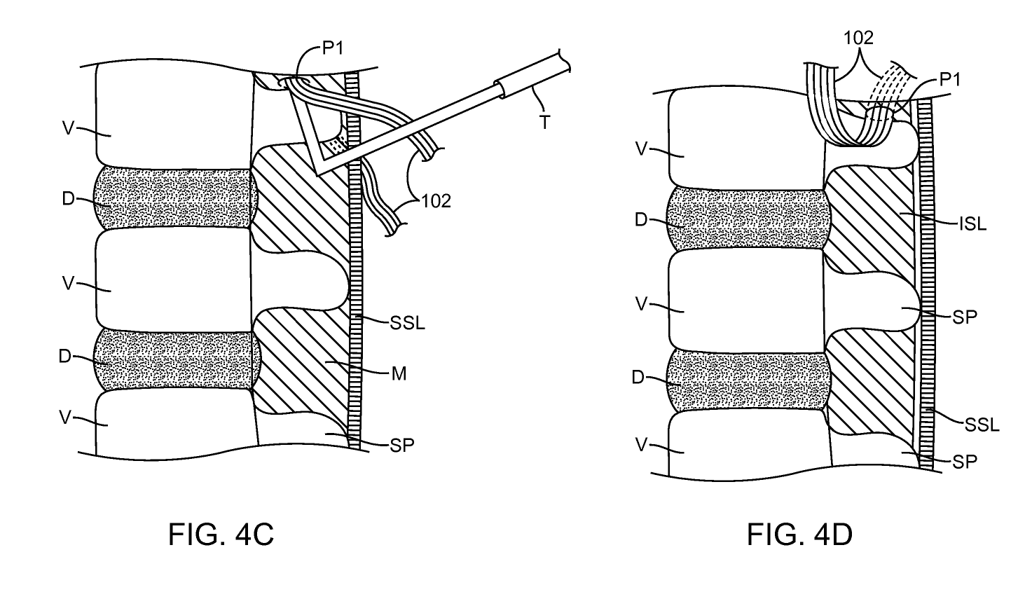

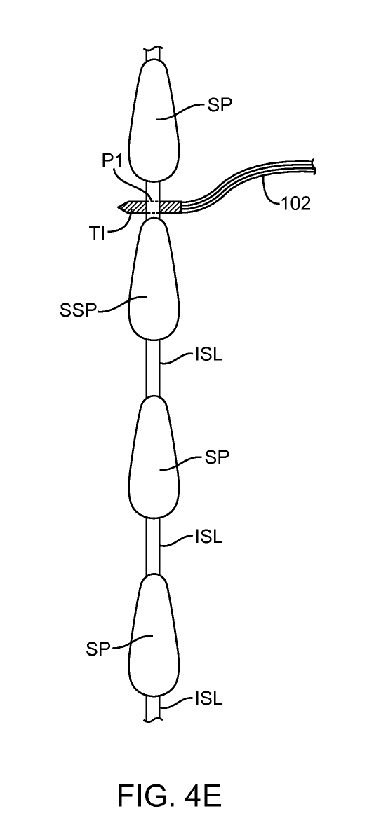

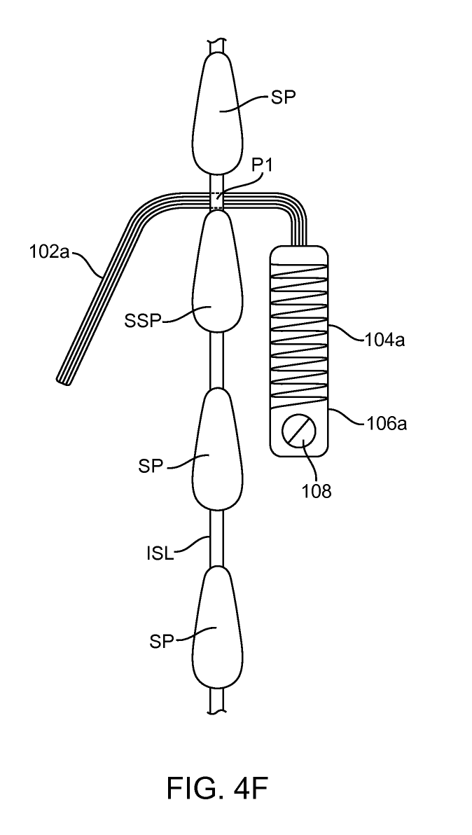

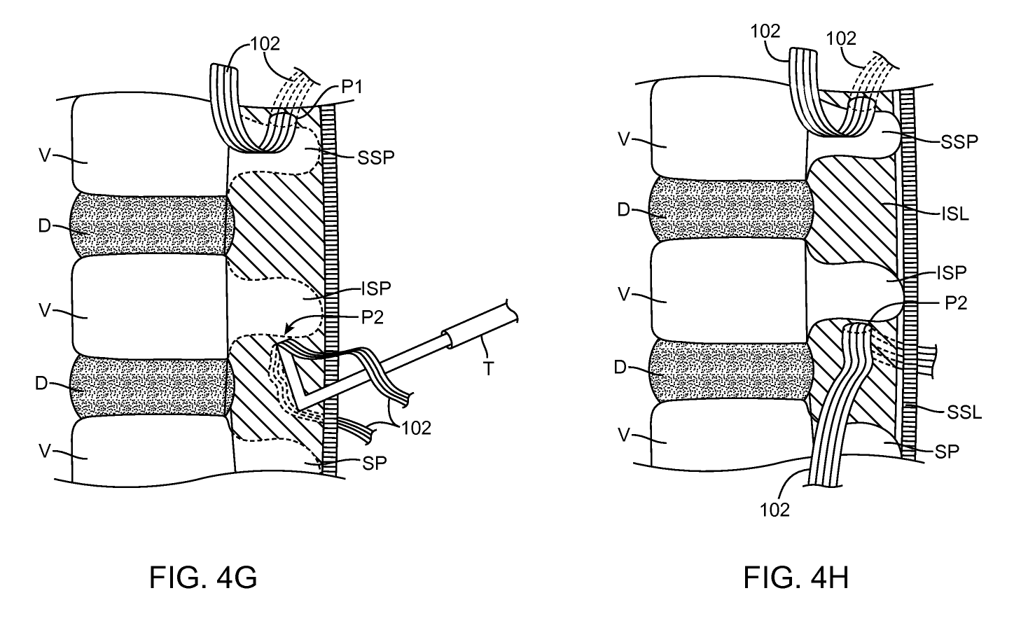

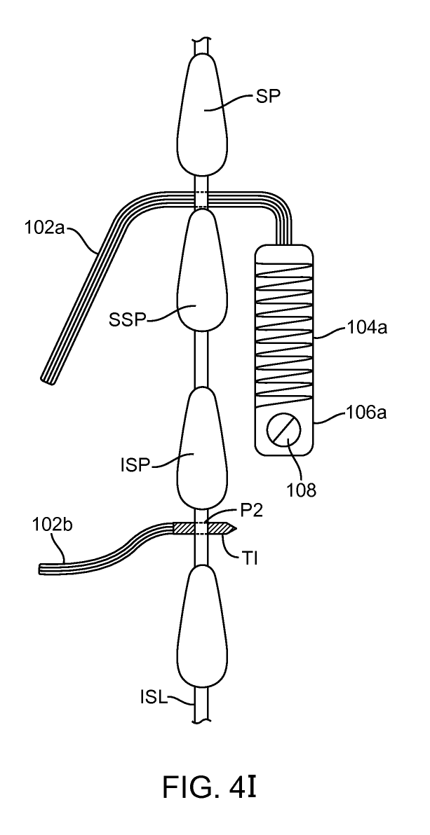

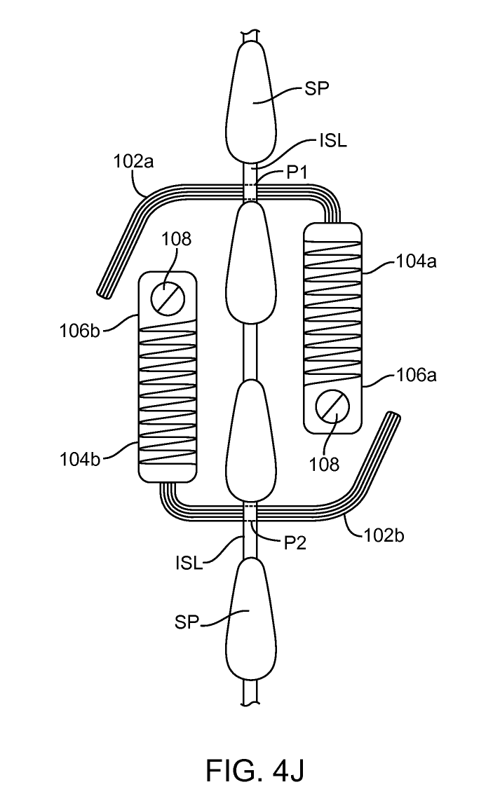

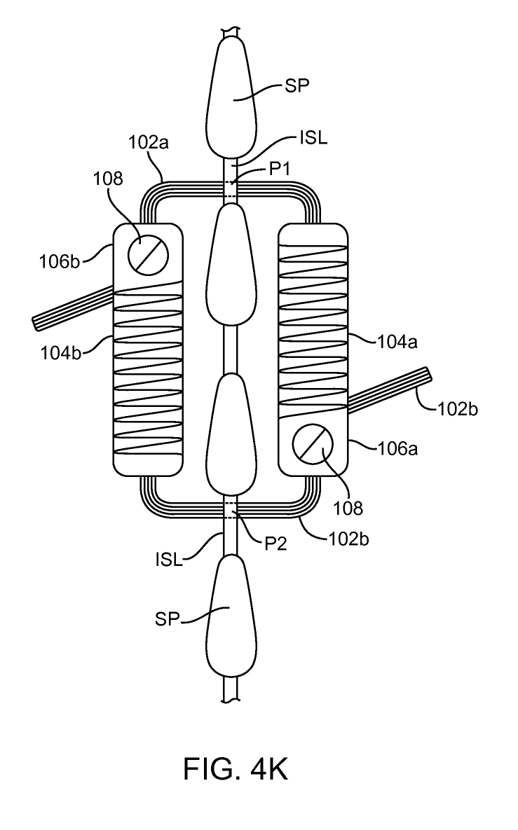

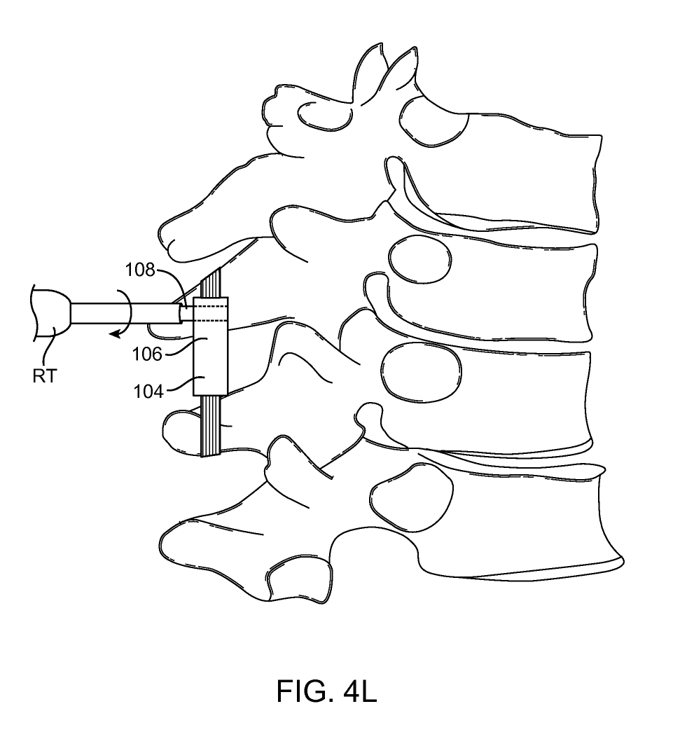

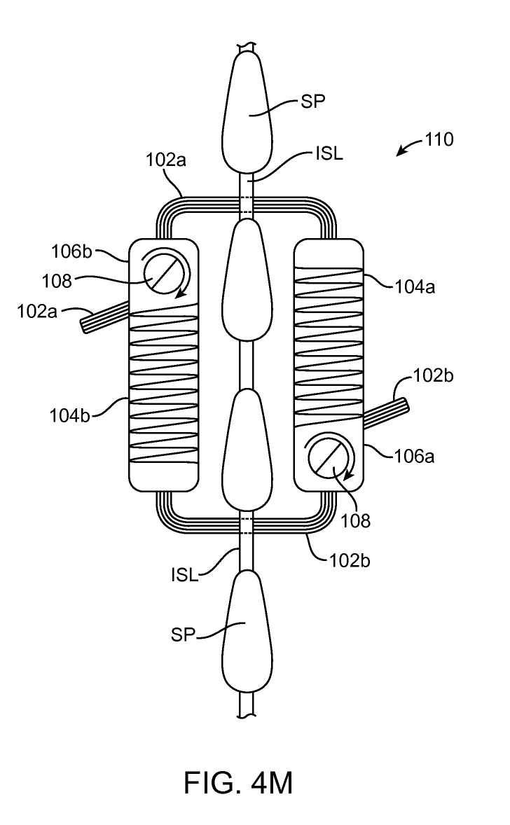

FIGS. 4A-4M illustrate an exemplary surgical method of implanting a spinous process constraint such as the embodiment of FIG. 2. One of the first steps to surgically implant a spinal implant is to make an incision to access the spinal area of interest. FIG. 4A shows the lumbar region of back K after an incision I has been made through the patient's skin. FIG. 4B illustrates the lumbar region of the spine after the incision I has been made through the patient's skin. Multifidus muscle and tendon M have been retracted with retraction tools TR to expose the spinous processes.

After the incision has been made, a piercing tool T having a sharp distal end may be used to access and pierce the interspinous ligament ISL while avoiding the supra spinous ligament SSL, creating an interspinous ligament perforation P1 superior of the first spinous process SSP of interest. This surgical approach is desirable since it keeps the supra spinous ligament intact and minimizes damage to the multifidus muscle and tendons and other collateral ligaments. As shown in FIG. 4C, from the right side of the spine, tool T accesses and pierces the interspinous ligament ISL adjacent of the first spinous process SSP of interest. The distal end of tool T is shown in dotted line. Alternatively, tool T may access and pierce the interspinous ligament ISL from the left side instead. The distal end of tool T is coupled with tether 102, parts of which are also shown in dotted line. In addition to accessing and piercing the interspinous ligament ISL, piercing tool T also advances or threads tether 102 through perforation P1. As shown in FIG. 4D, tool T is then removed, leaving tether 102 positioned through perforation P1. Multifidus tendon and muscle M is not shown in FIGS. 4C and 4D so that other elements are shown more clearly.

FIG. 4E is a posterior view of a section of the spine after the above steps have been performed. Often times, the distal tip TI of tool T is detachable. As shown in FIG. 4E, after tool T accesses and pierces the interspinous ligament ISL with distal tip TI, distal tip TI is detached from tool T and is left in place in perforation P1 (shown in dotted line) above the first spinous process SSP of interest. Tether 102 lags behind tip TI. In some cases, distal tip TI may fully pierce through interspinous ligament ISL. In these cases, distal tip TI has passed through the interspinous ligament ISL while a portion of tether 102 is left in place in perforation Pl.

After tip TI or a portion of tether TH is left in place in perforation Pl, another tool may couple with tip TI and pull tip TI such that it drags tether 102a and compliance element 104a to its appropriate position relative to the spine, as shown in FIG. 4F. Compliance element 104a is coupled to tether 102a and is used to provide a force resistive to flexion of spinous processes SP. Compliance element 104a includes a fastening mechanism or fastening element 106a and may further comprise a spring, a tensioning member, a compression member, or the like. Related compliance members are described in commonly owned U.S. patent application Ser. No. 12/106,103 (Attorney Docket No. 41564-705.201), the entire contents of which are incorporated herein by reference.

The steps of accessing the ISL, piercing the ISL, and threading tether 102 through a perforation are then repeated for the opposite, lateral side of the spine for an adjacent spinous process ISP, inferior of the first superior spinal process SSP of interest. As shown in FIGS. 4G and 4H, tool T accesses the interspinous ligament from the left side of the spinal midline and pierces the interspinous ligament ISL, creating a second perforation P2 located inferior of a second spinous process of interest, labeled as inferior spinous process ISP. As shown in FIG. 4G, the inferior spinous process ISP of interest is directly adjacent to and inferior of the first superior spinous process SSP of interest. However, it is entirely possible to perform the described procedure starting with the inferior spinous process ISP first instead of the superior spinous process SSP, for example, perforation P2 may be created before perforation P1. It is also possible that there may be a gap of one or more spinous processes SP between the spinous processes of interest. Multifidus tendon and muscle M is not shown in FIGS. 4G and 4H for clarity of the other shown elements.

As shown in FIGS. 4H, 4I and 4J, like with the steps shown in conjunction with the first piercing, tether 102b is pierced through perforation P2 and left in place along with distal tip TI of tool T (best seen in FIG. 4I). Another tool such as a pair of forceps, is then used to grasp distal tip TI to pull tether 102b and compliance element 104b in place relative to the spine, as shown in FIG. 4J. Opposing compliance members 104a and 104b on opposite sides of spinous processes SP are oriented in opposite directions. Each compliance element 104a, 104b is coupled with their respective tether 102a, 102b and has a respective fastening mechanism or fastening element 106a, 106b. Fastening mechanism 106a, 106b are configured to couple with the tether 102a, 102b of the opposing compliance member 104a, 104b. For example as shown in FIG. 4K, tether 102a is advanced through compliance member 104b and is coupled with fastening mechanism 106b while tether 102b is advanced through compliance member 104a and is coupled with fastening mechanism 106a. Except for their orientation, compliance members 104a and 104b are identical. One of skill in the art will appreciate that the tether may enter and exit the fastening mechanism in a number of different directions and configurations, and FIG. 4K merely is one exemplary embodiment.

Fastening mechanism 106 may comprise a driver feature 108. As shown in FIG. 4L, the driver feature is adapted to receive a rotating driver tool RT. The driver feature may be a Phillips head, a slotted flat head, a Torx head, a hex head, or the like. Rotation of tool RT, which may be either clockwise or counter-clockwise, changes the configuration of fastening mechanism 106 so as to lock and secure tether 102 in place. This forms a continuous, multi-component tether structure or constraint 110 which couples two spinous processes SP together, as shown in FIG. 4M. Compliance elements 104a, 104b are used to control flexion between spinous processes SP while tethers 102a, 102b and respective fastening mechanisms 106a, 106b contribute to coupling the spinous processes SP together. Depending on the location of the perforations P1 and P2 and the lengths of the compliance elements 104a, 104b, constraint 110 may couple more than two spinous processes SP together. In general, compliance elements 104a, 104b comprise spring-like elements which will elastically elongate as tension is applied through tethers 102a, 102b in an axis generally parallel to the spine. As the spinous processes or spinous process and sacrum move apart during flexion of the constrained spinal segment, the superior tether 102a and inferior tether 102b will also move apart. Compliance elements 104a, 104b each include spring-like elements which will elastically resist the spreading with a force determined by the mechanical properties of the spring-like element. Thus, constraint 110 provides an elastic resistance to flexion of the spinal segment beyond the neutral position. Constraint 110 is often configured to provide a resistance in the range from 7.5 N/mm to 20 N/mm but the resistance may be below 3 N/mm or even below 0.5 N/mm Constraint 110 may also be adjustable in certain dimensions to allow tightening over the spinous processes or spinous process and sacrum when the spinal segment is in a neutral position. Other, related tether embodiments and joining methods are disclosed in U.S. patent application Ser. No. 12/106,103 (Attorney Docket No. 41564-705.201), U.S. Patent Publication No. 2008/0009866 (Attorney Docket No. 41564-703.501), U.S. Patent Publication No. 2008/0108993 (Attorney Docket No. 41564-703.205), U.S. patent application Ser. No. 12/106,049 (Attorney Docket No. 41564-703.504) and U.S. Provisional Patent Application No. 60/936,897 (Attorney Docket No. 41564-705.101), each of which, the entire contents are incorporated herein by reference.

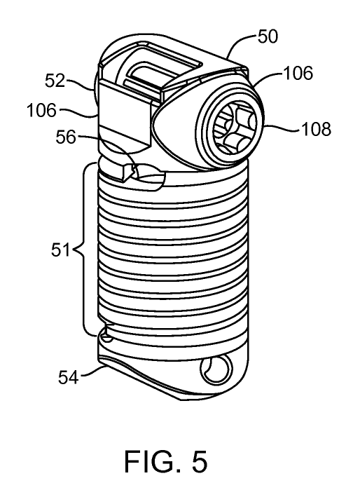

FIG. 5 illustrates an exemplary embodiment of a spring-like element 50 of compliance member 104a, 104b. Spring-like element 50 is generally similar to the spring-like elements disclosed in related, co-assigned U.S. patent application Ser. No. 12/106,103, the entire contents of which are incorporated herein by reference. Fastening mechanism 106 having a driver feature 108 is housed within spring-like element 50. Element 50 comprises a housing having a helical groove machined in the housing body to form the spring-like element. Element 50 includes an adjustable tether connector 52 and a fixed tether connector 54, both of which are preferably formed integrally or monolithically with the helical spring structure 51. Typically, the helical spring structure 51 and coupling portions of both tether connectors 52 and 54 will be formed from one piece of material, usually being a metal such as titanium, but optionally being a polymer, ceramic, reinforced glass or other composite, or other material having desired elastic and mechanical properties and capable of being formed into the desired geometry. In a preferred embodiment, spring-like element 50 is machined or laser cut from a titanium rod. Alternatively, a suitable polymeric material will be polyetherether ketone (PEEK). Other features may be built into the spring-like element 50, such as a stress relief hole 56. Components that compose the adjustable tether connector may potentially include a roller and a lock-nut; such components could be made from the same material as the element 50 and adjustable tether connector (e.g. titanium components if the spring-like element 50 is titanium), or they could be made from a different material (e.g. injection molded PEEK). The exterior of the spring-like element 50 may be covered with a protective cover, such as a sheath fabricated from an elastomer, polymer or other suitable material. The sheath may be placed over the body of the spring-like element 50 in order to prevent the intrusion of tissue and body fluids into the spaces between the turns of the coil and interior of the element.

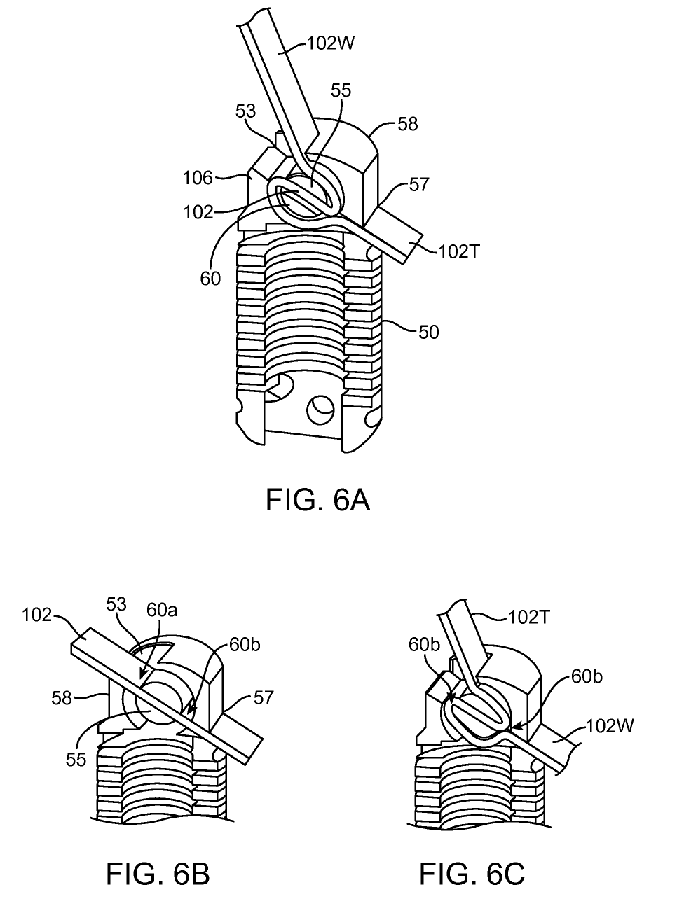

FIG. 6A shows a cross-section of spring-like element 50 having tether 102 locked therein. Tether 102 enters and exits the housing 58 of fastening mechanism 106 through entry aperture 53, then it passes through central channel 55, winds around roller 60 and the inside surface of housing 58, and finally exits through exit aperture 57. Roller 60 is housed within central channel 55 and is rotatable within tension element 50. Roller 60 is often substantially cylindrically shaped but may also have other shapes, for example, an eccentric shape. A round symmetrical roller will allow the tether 102 to spool evenly from both the working end and the tail end of the tether 102, while an eccentrically shaped roller will result in uneven spooling. The housing 58 of fastening mechanism 106 may be formed integrally with spring-like element 50 or may be separate.

During a procedure similar to the one described with reference to FIGS. 4A-4M, tether 102 is advanced through top aperture 53, central channel 55 and roller 60, and out through bottom aperture 57. As shown in FIG. 6B, top aperture 53, central channel 55, and bottom aperture 57 are aligned so permit easy passage of tether 102 therethrough. Roller 60 includes two side apertures 60a, 60b. Prior to the locking of the tether, entry aperture 53, side apertures 60a and 60b and exit aperture 57 are all aligned along a common axis. To provide such alignment, roller 60 may include an alignment feature such as a pin or shoulder. Thus, the roller 60 may be rotated until stopped by the pin or shoulder, thereby ensuring alignment of all the apertures. Once tether 102 is advanced through, roller 60 is rotated, via driver feature 108, thus creating a friction-based interference fit between roller 60, the inside surface of the housing and the tether 102. As shown in FIG. 6C, the fastening mechanism is rotated approximately 180° to create this fit. The rotation of the roller creates a tortuous path for the tether as it passes between side apertures 60a, 60b. The rotation may retract the working end 102w and tail end 102t of tether 102, sometimes of different lengths, inward toward roller 60. Offsetting roller 60 from its axis of rotation by using an eccentrically shaped roller changes the amount of tether drawn from either side. The roller may also be rotated a selected amount in order to draw a desired amount of the tether into the roller. For example, the roller may be rotated from about ¼ turn to two or more complete revolutions. Thus, not only will the locking mechanism secure the tether in position, but it may also be used to help adjust length or tension of the tether.

A friction-based interference fit is advantageous because the range along the tether to which the mechanism can attach is continuous, rather than in discrete increments of non-friction mechanisms such as teeth, hooks, loops, and the like. Thus, forces between roller 60 and tether 102 are distributed along a longer portion of tether 102. Additionally, high clamping forces are not required. Thus, the risk that any specific point of contact will abrade, wear, or will otherwise be damaged is minimized Furthermore, in contrast with other mechanisms that require high clamping forces, the discrete rotation of a tool is easier and more repeatable to perform during surgery.

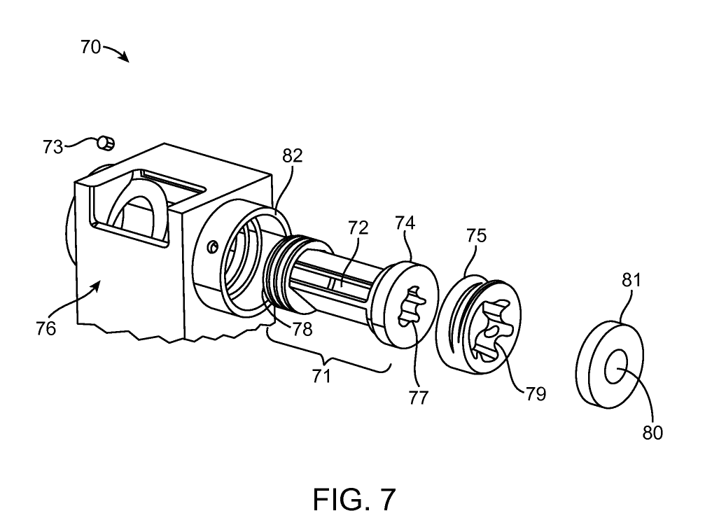

After the tether is secured, roller 60 is then locked in place. Various means may be provided to lock roller 60 in place within housing 58. Roller 60 and/or the inner surface of housing 108 may include male or female threads which engage the two elements together. The threads may be partially deformed, thereby helping to secure the roller element with the housing. Alternatively, a pin 73 may be coupled to housing 58 and roller 60 may comprise a groove adapted to receive pin 73. Another possibility is that housing 58 may include a flange adapted to retain roller 60. A set screw as described below with reference to FIG. 7 may also be provided to lock roller 60 in place. Rotation of roller 60 in the opposite direction unwinds tether 102 from roller 60 and reduces the interference fit. Roller 60 and/or housing 58 may further include a position indicator, such as detents or calibration marks, to provide visual, tactile, or audible feedback to an operator on the relative position of the roller with respect to housing 58.

FIG. 7 shows an exploded view of an exemplary fastening mechanism 70 that uses a locking set screw 75 to lock roller 76 in place. Roller 71 is generally similar to roller 60. It is positioned within housing 76 and includes slots 72 for a tether to be advanced through. Roller 71 has threads 78 on one end that may be threadably engaged with the housing 76. Roller 71 also has a shoulder 74 and includes driver features 77. Shoulder 74 is adapted to be engagable with locking set screw 75 and housing 76. After roller 71 has been rotated to lock and secure a tether in place, set screw 75 is set in a position to engage roller 71 with housing 76 and hold it in position relative to housing 76. Shoulder 74, set screw 75, and/or housing 76 have threads to allow such engagement. The threads may be partially deformed, thereby further securing the locking member with the housing. The threads prevent the roller 71 from unrolling thereby allowing release of the tether. Set screw 75 may comprise driver features 79 to allow rotation of the set screw. Driver features 77 of roller 71 and driver features 79 of set screw 75 each are adapted to receive a tool so as to permit rotation thereof. The driver features 77, 79 may be a Phillips head, a slotted flat head, a Torx head, a hex head, or the like. Driver features 79 of set screw 75 may comprise an aperture large enough to permit access to roller 71 with a tool permit rotation of roller 71 with a tool while set screw 75 is engaged with housing 76. An optional end cap 81 having a central aperture 80 may be positioned adjacent the set screw 75 and welded, bonded or otherwise affixed to the outer rim 82 of the housing 76 so as to capture all the components forming an inseparable assembly. The aperture 80 is sized to allow access to rotation of the set screw. This is desirable since it prevents parts from falling out during use and also provides a device which is easier to use since assembly is not required. In preferred embodiments, the assembly may not be disassembled without breaking or otherwise damaging the device. In other embodiments, the assembly may be disassembled without damaging the device.

One advantage of the roller locking mechanisms disclosed herein is that the tether is not deformed in planes in which it lies. The tether may be folded or rolled in a plane transverse to the planes in which it lies. This is desirable since it minimizes the possibility of twisting or tangling of the tether and also reduces wear and tear.

While the exemplary embodiments described above illustrate a fastening mechanism that is coupled with a spring-like compliance member, one will appreciate that the fastening mechanism may be used independently of a spring or other internal fixator. Other uses may include applications where a tether is secured with a knot, crimped or the like.

The flexion limiting device described above is a promising treatment for pain or instability related to flexion of the lumbar spine. It is easily implanted and adjusted. Implantation on the spinous processes is less invasive than fixation to other parts of the spinal anatomy, such as the pedicles or intervertebral disc space. However, in some cases, the tether straps may slide off the spinous processes or the straps may not be securely coupled and thus they may move. The process of passing the tether straps around the spinous processes and securing the straps to the compliance members may be cumbersome. Therefore, it would be desirable to provide such devices that overcome some of these challenges. Such devices preferably have improved attachment mechanisms for coupling with the spinous processes. The below describes several exemplary embodiments of a flexion limiting device that overcomes at least some of these challenges.

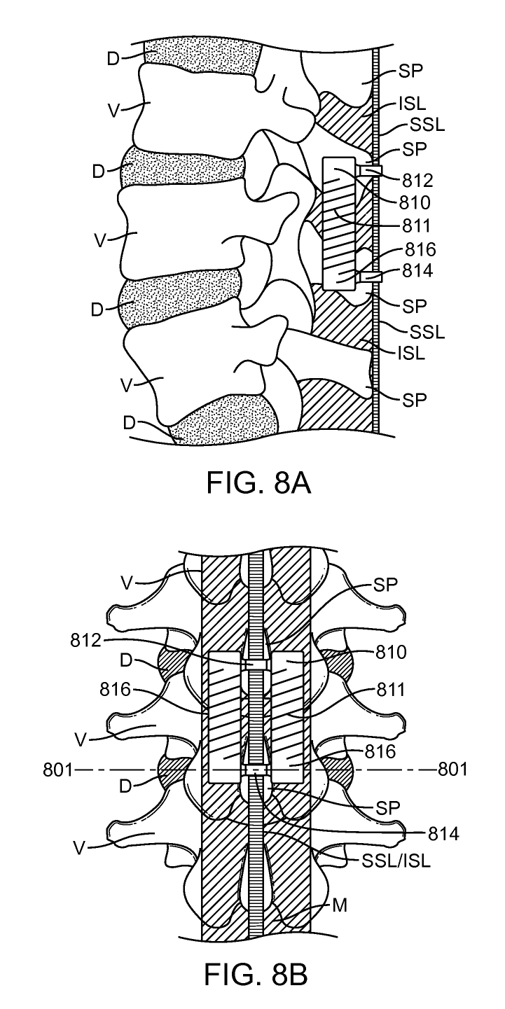

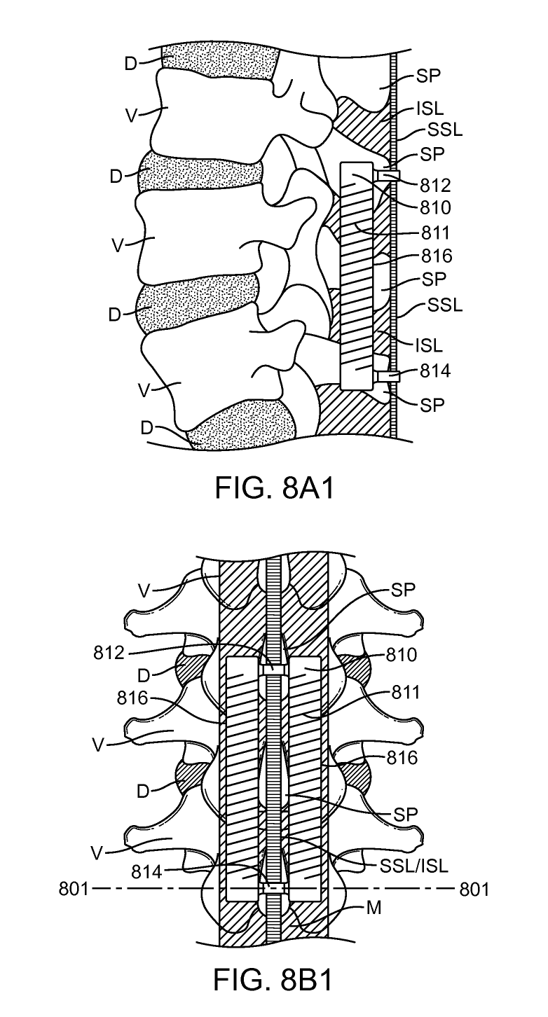

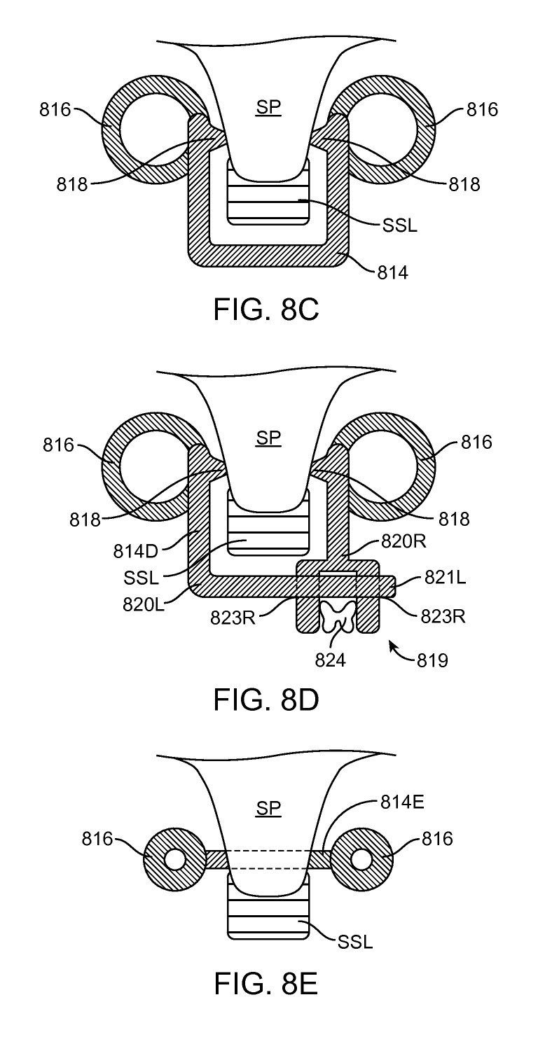

FIG. 8A is side view of an exemplary spinal implant 810 attached to a spine. FIG. 8B shows a posterior view of the same. In FIGS. 8A and 8B, various features of the spinal column are shown, including vertebral bodies V, intervertebral discs D, the interspinous ligament ISL, the supraspinous ligament SSL, spinous processes SP, and the multifidus tendon and muscles M. The spinal implant 810 comprises an upper attachment member 812, a lower attachment member 814, and two compliance members 816 disposed on either side of the spinal midline. The two compliance members 816 are each coupled to both the upper attachment member 812 and the lower attachment member 814. The upper attachment member 812 is securely fastened to a superior spinous process SP while the lower attachment member 814 is securely fastened to an inferior spinous process SP. Each attachment member 812, 814 is fastened over its respective spinal process SP such that the attachment member 812 or 814 is disposed over the dorsal aspect of the spinous process SP, as well as portions of the interspinous ligament ISL and supraspinous ligament SSL sharing the same transverse plane. The attachment members 812 and 814 will typically be securely fastened to the desired spinous process SP by being crimped or tightened over the desired spinal process SP and adjacent tissue without requiring substantial compromise or modification of the spinal anatomy (e.g., without requiring perforation of the interspinous ligament ISL, the multifidus tendon and muscles, or the like.) By securely fastening the attachment members 812 and 814 to their respective spinous process SP in such manner, damage to the spinal anatomy caused by having the spinal implant 810 implanted can be minimized Moreover, because the spinous processes SP are close to skin, implantation of the spinal implant 810 can minimally invasive and can generally avoid the compromise of spinal anatomy such as the facets, pedicles, disc, nerve roots, etc.

Each compliance member 816 typically includes an elastic or spring-like element, such as a spring or rubber block, such that the lower and upper ends of the compliance member 816 may be “elastically” or “compliantly” pulled apart as the attached spinous processes SP move apart during flexion. This elastic and/or compliant member may be internal or may be formed in the main body of the compliance member 816 (e.g., a spring cut into the middle portion of the main, hollow cylindrical body of the compliance member—see the windings 811 of each compliance member 816 shown in FIGS. 8A and 8B and the cross-sections of the compliance members 816 in FIGS. 8C-8D.) In this way, the spinal implant 810 provides an elastic force on the spinous processes SP which resists flexion. The force increases as the spinous processes move further apart. The elastic or spring-like elements of each compliance member 816 will elastically resist the spreading of the superior spinous process SSP and the inferior spinous process ISP with a force determined by the mechanical properties of the elastic or spring-like element. Thus, constraint or spinal implant 810 provides an elastic resistance to flexion of the spinal segment beyond the neutral position. Constraint 810 will typically be configured to provide a resistance in the range from 7.5 N/mm to 25 N/mm, and preferably 10 N/mm to 15 N/mm By applying a very low elastic resistance to flexion of the spinal segment in such ranges, effective pain relief can be achieved with minimum risk of damage to the spinous processes and other vertebral and spinal structures which could result from restriction with relatively rigid structures and even elastic structures with higher elastic resistances Constraint 810 may also be adjustable in certain dimensions to allow tightening over the spinous processes or spinous process and sacrum when the spinal segment is in a neutral position. Other, related tether embodiments and joining methods are disclosed in U.S. patent application Ser. No. 12/106,103 (Attorney Docket No. 41564-705.201), U.S. Patent Publication No. 2008/0009866 (Attorney Docket No. 41564-703.501), U.S. Patent Publication No. 2008/0108993 (Attorney Docket No. 41564-703.205), U.S. patent application Ser. No. 12/106,049 (Attorney Docket No. 41564-703.504) and U.S. Provisional Patent Application No. 60/936,897 (Attorney Docket No. 41564-705.101), each of which, the entire contents are incorporated herein by reference.

Each compliance member 816 may also be laterally flexible so that axial rotation and lateral bending of the spine are generally unrestricted. The compliance members 816 may be formed monolithically with the attachment members 812 as illustrated in FIG. 8D, or may be distinct components attached to the attachment members 812 with a connector 819 as illustrated in FIG. 8C. The connector may be rigid, such as a weld or rigid fastener, or may allow one or more degrees of freedom, such as a pin or ball joint.

As shown in FIGS. 8A and 8B, there are no other spinal processes SP between the upper attachment member 812 and the lower attachment member 814. In other embodiments, the size of the compliance members 816 and/or the placements of the upper attachment member 812 and the lower attachment member 814 may be selected such that there are one or more gaps of spinal processes SP between the upper attachment member 812 and the lower attachment member 814 or a gap of more than one spinal process SP. For example, FIG. 8A1 shows a side view of a spinal implant 810 having an axial length selected such that there is a gap of one spinal process SP between the upper attachment member 812 and the lower attachment member 814; FIG. 8B1 shows a posterior view of the same. Mechanisms for adjusting the length of or tension provided by the compliance member 816 may also be provided. For example, each compliance member 816 may comprise an upper element, a lower element, and an elastic block or band therebetween having an adjustable length. The connector 819 may provide a mechanism for adjusting the length or tension by allowing attachment members 812 to connect to a range of locations along compliance members 816, or conversely by allowing compliance members 816 to attach to a range of locations along attachment members 812.

An exemplary procedure of implanting the spinal implant 810 may include steps of first fastening the upper attachment member 812 to a superior spinal process SP and the lower attachment member 814 to an inferior spinal process SP. The compliance members 816 may be pre-attached to the attachment members 814, or if the compliance members 816 are provided as separate components the procedure may then include the step of joining the compliance members 816 to the attachment members 812, 814 on either side of the spinal midline (e.g., screwing the top portion of a compliance member 816 to the side of the upper attachment member 812 and screwing the bottom portion of the same compliance member 816 to the side of the lower attachment member 814; screw holes will typically be provided for each of the aforementioned component parts.)

FIGS. 8C-8E show partial cross-sectional views of various embodiments of the attachment member of the spinal implant 810. The cross-sectional views are taken along line 801 in FIG. 8B. Various features may be provided to the attachment member 812 and 814 to improve their ability to fasten onto the spinous processes. For example, the inner surfaces of the attachment member 812 and 814 may be treated (e.g., plasma bead spray, hydroxyapatite, etc.) to facilitate osseointegration with the spinous processes. Also, as shown in FIG. 8C, the attachment member 814 may include one or more protrusions or spikes 818 facing the spinous process SP to facilitate the fastening of the attachment member 814 to the spinal process SP. The attachment member 814 shown in FIG. 8C is monolithic and is connected to compliance members 816 through connectors 819A. The attachment member 814 may be resilient and biased so that it can be expanded before placement over the spinal process SP; a spring force in the attachment member 814 would cause it to clip onto the spinal process SP when the attachment member 814 is no longer kept expanded. Alternatively or in combination, the attachment member 814 may be deformable and be crimped onto the spinal process SP. In other embodiments, an attachment member may be an assembly that comprises more than one distinct part. For example, as shown by FIG. 8D, an attachment member 814D may be an adjustable clamping mechanism comprising a left portion 820L and a separate right portion 820R which may be fixedly coupled together. The left portion 820L includes an arm 821L and the right portion 820R includes a fastener 819. The arm 821L of the left portion 820L may be slid through the apertures 823R of the fastener 819 of the right portion 820R. The arm 821L may be tightened to securely couple with the fastener 819 with set screw 824 when the left portion 820L and right portion 820R are at a desired distance from each other. In still other embodiments, for example as shown in FIG. 8E, the attachment member 814E may be a screw, bolt, pin, or the like which is passed directly through the spinal process SP. Elements of the embodiments illustrated in FIGS. 8C-8E may be combined. For example, the resilient, biased attachment member 814 of FIG. 8C may comprise an adjustable clamping mechanism as in FIG. 8D, such that the attachment member conforms to the spinous process when the clamping mechanism is secured, for better purchase on the bone of the spinous process. The attachment member 814E of FIG. 8E may additionally comprise an adjustable clamping mechanism as in FIG. 8D, to adjust for the width of the spinous process or otherwise secure the compliance elements snugly against the spinous processes.

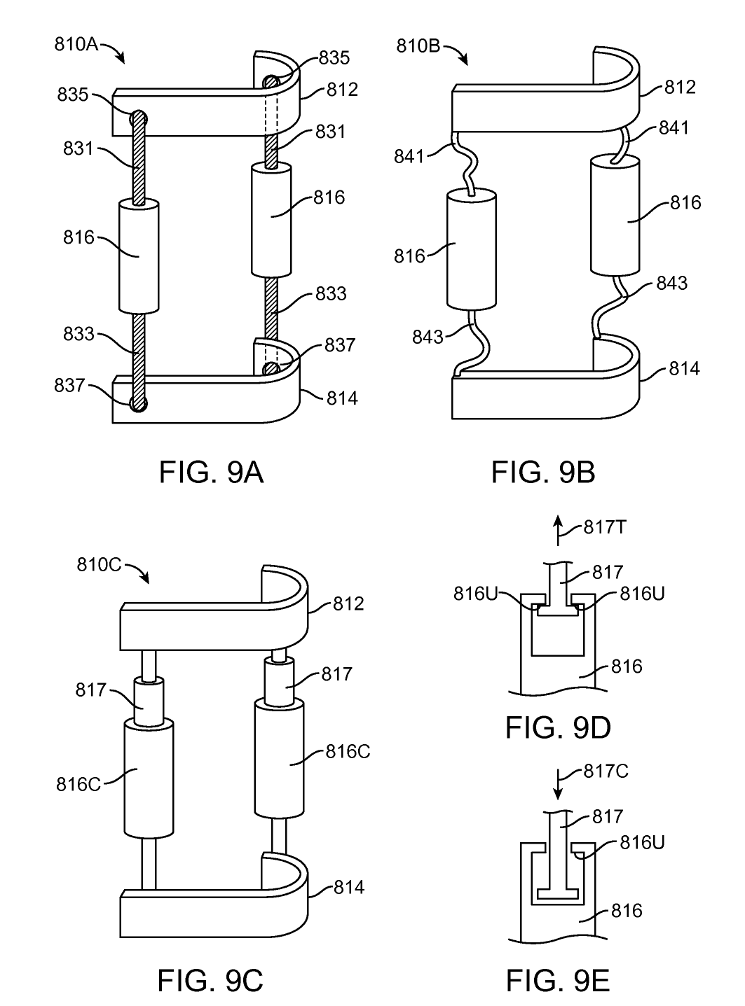

FIGS. 9A-9C illustrate exemplary spinal implants according to various embodiments of the invention. The spinal implants shown in FIGS. 9A-9C are generally similar to the spinal implant 810 described above. The spinal implants shown in FIG. 9A-9C have their compliance members 816 coupled to their respective attachment members 812 and 814 such that certain loads are prevented from being transmitted to the compliance members 816. The attachment members 812 and 814 shown in FIGS. 9A-9C can be fastened or fixedly secured to their respective spinal processes SP in any number of ways including those described above in reference to FIGS. 8A-8E.

FIG. 9A shows a spinal implant 810A. The compliance members 816 of the spinal implant 810A are coupled to the upper attachment member 812 through upper connectors 831 and coupled to the lower attachment member 814 through lower connectors 833. The upper connector 831 and the lower connector 833 will typically be non-compliant shafts. Universal joints 835 attach the upper connector 831 to the upper attachment member 812. Pivots such as universal joints 837 attach the lower connector 833 to the lower attachment member 814. The universal joints 835 and 837 may be ball joints, Cardan joints, or a flexible elastomeric coupling (e.g., a Boge joint.) The universal joints 835 and 837 prevent complex loads such as bending, torsional and shear loads, from being transferred to the compliance members 816 while allowing tensile loads to be transferred to the compliance members 816. The pivots may also be simple pin joints if only one degree of freedom is needed.

FIG. 9B shows a spinal implant 810B. The compliance members 816 of the spinal implant 810B are coupled to the upper attachment member 812 through flexible upper tether connectors 841 and coupled to the lower attachment members 814 through flexible lower tether connectors 843. By using flexible tethers to join the compliance members 816 to the attachment members 812 and 814, compressive loads are not transferred to the compliance members 816 while tensile loads can be transmitted. The tether connectors 841, 843 can also act as a universal joint described above and prevent bending and torsional loads from being transmitted to the compliance members 816.

FIG. 9C shows a spinal implant 810C. The compliance members 816C of the spinal implant 810C are directly attached to the upper attachment member 812 and lower attachment member 814. The compliance members 816C also have a telescoping mechanism, including a telescoping column 817, which also prevents compressive loads from being transmitted to the compliance members 816 while tensile loads can be transmitted. FIGS. 9D-9E show schematic side views of the telescoping mechanism. As shown in FIG. 9D, when compliance member 816 is in axial tension, the bottom portion of the telescoping column 817 abuts the upper portion of the main body of the compliance member 816 such that the tensile force is transmitted to the main body. As shown in FIG. 9E, the telescoping mechanism collapses under compression such that compressive loads are not transmitted to the attachment member 816. The telescoping mechanism of FIG. 9D may be combined with the pivots of FIG. 9A to achieve rotational and translational degrees of freedom.

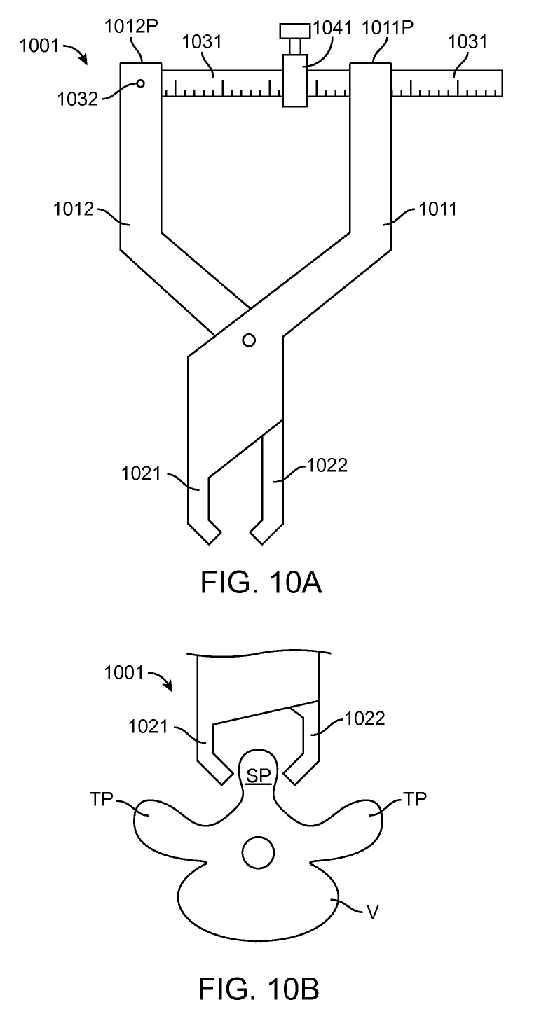

As discussed above, an attachment member 812 or 814 may be deformable and crimped onto a desired spinal process SP. Devices and methods can be provided such that the attachment member 812 or 814 is deformed in a controlled manner such that there will not be any substantial damage to the bone (e.g., by avoid crushing the bone or minimizing undesired bone contusions) while at the same time ensuring a robust interface between the bone and the attachment member 812 or 814. FIG. 10A shows a hand-holdable spinous process measurement and crimping tool 1001. The measurement and crimping tool 1001 will typically be in the form of pliers. As shown in FIG. 10A, the tool 1001 comprises a first handle or arm 1011, a second handle or arm 1012, a first jaw 1021, a second jaw 1022, a ruler 1031 pivotably coupled with the second arm 1012 through pivot 1032 and threaded through the proximal end of the first arm 1011, and a moveable stop 1041 coupled to and slideable over the ruler 1041 and disposed between the proximal end 1011P of the first arm 1011 and proximal end 1012P of the second arm 1012. As shown in FIG. 10B, the tool 1001 can first be used to measure the width of a spinal process SP. The maximum desired displacement of the to-be-crimped attachment member 812 or 814 can then be determined. This maximum desired distance is one in which the attachment member 812 or 814 is crimped such that bone-crushing is avoided while at the same time a robust interface between the bone and attachment member 812 or 814 is ensured. This maximum desired distance may be, for example, slightly less than the sum of the measured thickness of the spinal process and the thickness of the attachment member 812 or 814. The moveable stop 1041 can then accordingly be set a predetermined distance from the proximal end 1012P of the second arm such that the jaws 1021 and 1022 can only be brought together up to a desired distance corresponding to the maximum desired displacement of the to-be-crimped attachment members 812 or 814. Thus, the same tool 1001 can be used to both measure bone thickness of the spinal process SP and to crimp an attachment member 812 or 814. Alternatively, separate tools may be provided for measurement and crimping.

The following US patents and applications also disclose features that may be used alone or in combination with any of the embodiments described above: 60/936,897 (Attorney Docket No. 41564-705.101); Ser. No. 12/106,103 (Attorney Docket No. 41564-705.201); Ser. No. 12/535,560 (Attorney Docket No. 41564-705.501); U.S. Pat. No. 7,458,981 (Attorney Docket No. 41564-704.201); Ser. No. 12/262,877 (Attorney Docket No. 026398-000220US); U.S. Pat. No. 8,105,353 (Attorney Docket No. 41564-704.301); and Ser. No. 12/426,167 (Attorney Docket No. 41564-703.501); the entire contents of each of which is incorporated herein by reference.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A device for limiting flexion of a spine, said device comprising:
at least one compliance member;
an upper fastener configured to be fixedly coupled to a superior spinous process; and
a lower fastener configured to be fixedly coupled to an inferior spinous process,
wherein the at least one compliance member is coupled to the upper and lower fasteners such that the at least one compliance member provide a force resistant to flexion between the upper and the lower spinous processes.

2. The device of claim 1, wherein the at least one compliance member comprises a first compliance member and a second compliance member, the second compliance member being configured to be disposed across a spinal midline, wherein the first compliance member and the second compliance member are each coupled to both the upper and lower fasteners.

3. The device of claim 1, wherein one or more of the compliance members comprises an elastic member configured to provide an elastic tension between the superior spinous process and inferior spinous process to resist flexion therebetween.

4. The device of claim 3, wherein the elastic member comprises a spring.

5. The device of claim 4, wherein one or more of the compliance members comprises a hollow, cylindrical main body and the elastic member comprises a spring cut into the cylindrical main body.

6. The device of claim 1, wherein one or more of the compliance members are laterally flexible so that axial rotation and lateral bending of the spine are generally unrestricted when the upper fastener is coupled to the superior spinous process, the lower fastener is coupled to the inferior spinous process, and the pair of compliance members are coupled to the lower and upper fasteners.

7. The device of claim 1, wherein the axial length of device can be adjusted.

8. The device of claim 7, wherein the axial length of the device can be adjusted by adjusting the axial length of the at least one compliance member.

9. The device of claim 1, wherein the upper fastener is monolithic and resilient such that the upper fastener can be clipped onto the superior spinous process.

10. The device of claim 1, wherein the lower fastener is monolithic and resilient such that the lower fastener can be clipped onto the inferior spinous process.

11. The device of claim 1, wherein the upper fastener comprises an interior surface configured to face an exterior surface of the superior spinous process and one or more protrusions disposed on an interior surface of the upper fastener, the one or more protrusions being adapted to facilitate fixed attachment of the upper fastener to the superior spinous process.

12. The device of claim 1, wherein the lower fastener comprises an interior surface configured to face an exterior surface of the inferior spinous process and one or more protrusions disposed on an interior surface of the lower fastener, the one or more protrusions being adapted to facilitate fixed attachment of the lower fastener to the inferior spinous process.

13. The device of claim 1, wherein the upper fastener comprises an interior surface treated to facilitate osseointegration with the superior spinous process.

14. The device of claim 1, wherein the lower fastener comprises an interior surface treated to facilitate osseointegration with the inferior spinous process.

15. The device of claim 1, wherein the upper fastener comprises a screw, bolt, or pin configured to be passed through the superior spinous process.

16. The device of claim 1, wherein the lower fastener comprises a screw, bolt, or pin configured to be passed through the inferior spinous process.

17. The device of claim 1, wherein the upper fastener comprises an adjustable clamping mechanism configured to be tightened over the superior spinous process.

18. The device of claim 1, wherein the lower fastener comprises an adjustable clamping mechanism configured to be tightened over the inferior spinous process.

19. The device of claim 1, wherein the device is adapted to prevent compressive loads from being transferred to the at least one compliance member while tensile loads are allowed to be transferred thereto.

20. The device of claim 1, wherein one or more of the at least one compliance member comprise a telescoping column configured to prevent compressive loads from being transmitted to the compliance members while allowing tensile load to be transmitted thereto.

21. The device of claim 1, further comprising one or more connectors configured to couple one or more of the at least one compliance member to the upper fastener or lower fastener.

22. The device of claim 20, wherein the one or more connectors comprise one or more flexible tether structures, the tether structures coupling the one or more of the at least one compliance member to the upper fastener or lower fastener such that compressive load are prevented from being transferred to the at least one compliance member while tensile loads are allowed to be transferred thereto.

23. The device of claim 21, wherein the one or more flexible tether structures are further configured to prevent bending loads or torsional loads from being transmitted to the compliance members.

24. The device of claim 22, wherein the one or more connectors comprise one or more non-compliant shafts.

25. The device of claim 20, wherein the one or more connectors are coupled to the upper or lower fasteners through one or more universal joints, the one or more universal joints being configured to prevent bending, torsional, or shear loads from being transferred to the compliance members while allowing tensile loads to be transferred to the compliance members.

26. The device of claim 1, wherein the at least one compliance member provides an elastic resistance to flexion in a range from 7.5 N/mm to 20 N/mm.

27. The device of claim 26, wherein the at least one compliance member provides an elastic resistance to flexion in a range from 10 N/mm to 15 N/mm.

28. A method for restriction flexion of a spine, said method comprising:
fixedly attaching an upper fastener to an upper spinous process; and
fixedly attaching a lower fastener to a lower spinous process,
wherein at least one compliance member is attached to both the upper fastener and lower fastener; and
resisting flexion between the upper and lower spinous processes, wherein the at least one compliance member provides a force resistant to the flexion.

29. The method of claim 28, wherein the at least one compliance member is fixedly attached to the upper and lower fasteners during a surgical procedure.

30. The method of claim 29, wherein the at least one compliance member is fixedly attached to the upper and lower fasteners after the upper fastener has been fixedly attached to the upper spinous process.

31. The method of claim 29, wherein the at least one compliance member is fixedly attached to the upper and lower fasteners after the lower fastener has been fixedly attached to the lower spinous process.

32. The method of claim 28, wherein the at least one compliance member is fixedly attached to the upper and lower fasteners prior to a surgical procedure.

33. The method of claim 28, wherein the at least one compliance member comprises a first compliance member and a second compliance member, the first and second compliance members being disposed on opposite sides of a spinal midline.

34. The method of claim 28, wherein there are no other spinous processes between the upper and lower spinous processes.

35. The method of claim 28, wherein there are one or more spinous processes between the upper and lower spinous processes.

36. The method of claim 28, further comprising preventing bending, torsional, or shear loads from being transferred to the compliance members while allowing tensile loads to be transferred to the compliance members.

37. The method of claim 28, wherein attaching the upper fastener to the upper spinous process comprises crimping the upper fastener over the upper spinous process such that the upper fastener is fixedly secure to the upper spinous process.

38. The method of claim 28, wherein attaching the upper fastener to the upper spinous process comprises crimping the lower fastener over the lower spinous process such that the lower fastener is fixedly secure to the lower spinous process.

39. The method of claim 28, wherein the upper fastener comprises an adjustable clamping mechanism and attaching the upper fastener to the upper spinous process comprises placing the upper fastener over the upper spinous process and tightening the adjustable clamping mechanism such that the upper fastener is fixedly secure to the upper spinous process.

40. The method of claim 28, wherein the lower fastener comprises an adjustable clamping mechanism and attaching the lower fastener to the lower spinous process comprises placing the lower fastener over the lower spinous process and tightening the adjustable clamping mechanism such that the lower fastener is fixedly secure to the lower spinous process.

41. The method of claim 28, wherein attaching the upper fastener to the upper spinous process comprises expanding the upper fastener, placing the expanded upper fastener over the upper spinous process, and releasing the upper fastener from expansion so that a spring force in the upper fastener causes the upper fastener to clip over the upper spinous process.

42. The method of claim 28, wherein attaching the lower fastener to the lower spinous process comprises expanding the lower fastener, placing the expanded lower fastener over the lower spinous process, and releasing the lower fastener from expansion so that a spring force in the lower fastener causes the lower fastener to clip over the lower spinous process.

43. The method of claim 28, wherein resisting flexion between the upper and lower spinous processes comprises providing an elastic resistance to flexion in a range from 7.5 N/mm to 20 N/mm.

44. The method of claim 43, wherein providing an elastic resistance to flexion in a range from 7.5 N/mm to 20 N/mm comprises providing an elastic resistance to flexion in a range from 10 N/mm to 15 N/mm.

45. The method of claim 28, further comprising:
providing a thickness measurement and crimping tool, the tool comprising a pair of handles, a pair of jaws, a ruler disposed between proximal ends of the pair of handles, and a moveable stop disposed on the ruler;
clenching the pair of jaws of the tool over the upper spinous process to measure the thickness of the upper spinous process;
setting a first maximum closure distance of the jaws by adjusting the location of the moveable stop on the ruler, the first maximum closure distance corresponding to the measured thickness of the upper spinous process;
positioning the upper fastener over the upper spinous process; and
crimping the upper fastener with the pair of jaws of the tool such that the upper fastener is fixedly secure to the upper spinous process without substantial damage to the bone of the upper spinous process.

46. The method of claim 45, further comprising:
clenching the pair of jaws of the tool over the lower spinous process to measure the thickness of the lower spinous process;
setting a second maximum closure distance of the jaws by adjusting the location of the moveable stop on the ruler, the second maximum closure distance corresponding to the thickness of the lower spinous process;
positioning the lower fastener over the spinous process; and
crimping the lower fastener with the pair of jaws of the tool such that the lower fastener is fixedly secure to the lower spinous process without substantial damage to the bone of the lower spinous process.

47. A system for limiting flexion of a spine, said system comprising:
the device of claim 1; and
a thickness measurement and crimping tool, said tool comprising a pair of handles, a pair of jaws configured for measuring the thickness of a spinous process, a ruler disposed between proximal ends of the pair of handles, and a moveable stop disposed on the ruler and configured to limit the closure distance of the pair of jaws such that the lower or upper fastener of the device can be crimped over the inferior or superior spinous process by the pair of jaws without substantial damage to the bone of said spinous process.
